(19) United States
(12) Patent Application Publication
(10) Pub. No.: US 2016/0135694 A1
(43) Pub. Date: May 19, 2016

(54) MEDICAL RADAR METHOD AND SYSTEM

(71) Applicant: NEDERLANDSE ORGANISATIE VOOR TOEGEPAST- NATUURWETENSCHAPPELIJK ONDERZOEK TNO, 's-Gravenhage (NL)

(72) Inventor: Philip van Dorp, 's-Gravenhage (NL)

(21) Appl. No.: 14/896,434

(22) PCT Filed: Jun. 6, 2014
(86) PCT No.: PCT/NL2014/050371

§ 371 Date: Dec. 7, 2015

(30) Foreign Application Priority Data
Jun. 7, 2013 (EP) 13171078.2

(51) Int. Cl. A61B 5/02; A61B 5/00

(52) U.S. Cl. CPC A61B 5/02028; A61B 5/7275; A61B 5/7207; A61B 2562/0228

(57) ABSTRACT

Radar is used to measure internal body motion. Radar reflections from the body are measured using a range of frequencies that includes a higher frequency band and a lower frequency band. In the higher frequency band, e.g. above 24 GHz, the radar signal hardly penetrates the skin, whereas it penetrates deeper into the chest in the lower frequency band e.g. below 10 GHz. Chest surface motion is estimated by means of the measurements using the higher frequency band. Effects of the estimated chest surface movement are subtracted from the measurements from the lower frequency band. The resulting response after removal is used to fit a model of a heart. Fitting may be performed in a series of fitting steps, including fitting parameters X of a geometric model to the measurements; determining a least square solution of fit errors between the measurements and a Taylor expansion from the grid model obtained with the fitted parameters X as a function of adaptions of the grid model; and subsequently determining a further adaptation of the grid model that best fits the measurements.

### FIELD OF THE INVENTION

The invention relates to a medical radar method and system for measuring parameters of a human body using radar reflection.

### BACKGROUND

From EP 2368492 it is known to measure human heart performance using radar techniques. The document discloses that the radar frequency can be chosen so that the radar signals penetrate the human body, in an interval of 2.4 GHz and circa 5.6 GHz for example. A model is used that predicts synthetic radar data as a function of parameters of the heart, such as heart beat frequency, amplitude and phase, as well as heart volume, blood perfusion etc. Actual radar is measured and values of these parameters are selected that make the model generate synthetic radar data best fit the actual radar data.

EP 2368492 uses a frequency modulated continuous wave (FMCW) radar system. Herein the reflected radar signal is mixed with the transmitted FMCW signal, which results in a beat signal that shows patterns of Doppler distribution and velocity distribution that are indicative for the heart performance. The fitting process can be performed either in the time domain or in the spectral domain. The former represents the output of the radar system for a grid of time points and the latter represents it for a grid of frequency points. The heart frequency grid lies in the interval 0.7-1.7 Hz. Inverse determination of heart parameters from time domain and spectral domain representations are termed an inverse time and an inverse frequency approach respectively, whose accuracies are compared. Similar results are disclosed in an article by Laura Anitori et al, titled “FMCW radar for life-sign detection”, published in the proceedings of the IEEE radar conference 2009 pages 1-6.

EP 2368492 notes that respiration can affect the resulting estimate of heart volume. The document proposes various solutions to this problem such as high pass filtering with a transition frequency of 0.7 Hz. The effect of respiration on the results of different algorithms for estimation heart parameters is compared, which shows that an inverse time algorithm suffers least. The inverse time algorithm suffers least from the effect of respiration on the estimation of heart volume. Anitori et al also note that heartbeat frequency determination is not as simple as the determination of respiration rate. To address this problem they propose to select bins in the spectral representation that most clearly show heartbeat. In an autocorrelation approach (time domain) they propose to apply high pass filtering to pass frequencies above 0.8 Hz and using the signal in time intervals where heartbeat is expected.

### SUMMARY

Among others, it is an object to provide for an improved method and system to measure parameters of a human body using radar signal reflection.

A method of measuring internal body motion using radar is provided wherein the method comprises
measuring radar reflections using a range of frequencies that includes a higher frequency band and a lower frequency band;
estimating chest surface motion using the measurements using the higher frequency band;
removing effects of the estimated chest surface movement from the measurements from the lower frequency bands. It has been found that in higher frequency bands of e.g. frequencies above 24 GHz the radar signal does not or hardly penetrate through the skin. In lower frequency bands, of e.g. below 10 GHz, the radar signal does at least partly penetrate, but due to bandwidth limitations the responses of the skin and the heart are hard to separate. By using measurements obtained using a high frequency band to control removal of a chest surface response component from the response to a low frequency band, the heart response can be better identified in the low frequency band. Removing effects of the estimated chest surface movement means that the signal components in the signal due to the chest surface are reduced relative to components due to chest-internal effects. Complete reduction to zero is not needed. In an embodiment, the higher radar frequency band may be a 24Ghz band, 60 GHz band or 76 Ghz band and the lower radar frequency band may be a 2-10 Ghz band, for example a band of 2.4 to 3 GHz.

The results may be used an fitting process to fir a model of a heart to the measurements. It has been found that the removal of the chest surface response component prior to fitting significantly improves the correspondence between the results of the fit and reality.

In an embodiment a radar system for is provided for monitoring internal motion within the chest of a body, the radar system comprising a radar signal generator coupled to a transmission antenna and cause transmission of radar signals in a range of frequencies that includes a higher frequency band and a lower frequency band;
at least one receiver for receiving radar reflections in the higher frequency band and the lower frequency band;
a signal processing system configured to estimate chest surface motion using the measurements using the higher frequency band and to remove effects of the estimated chest surface movement from the measurements from the lower frequency bands. The receiver may comprise a radar reception antenna or input for coupling to the reception antenna and a circuit coupled to the reception antenna or input, for detecting reflections of the transmitted radar signal. The circuit may contain a mixer coupled to the radar reception antenna or the input for example.

### BRIEF DESCRIPTION OF THE DRAWING

These and other objects and advantageous aspects will become apparent from a description of exemplary embodiments with reference to the following figures.

FIG. 1 shows a radar system;

FIGS. 1a-c show aspects of a radar system;

FIG. 2 illustrates a transmitted FMCW signal and its echo;

FIG. 3 shows a flow-chart of multi frequency signal processing;

FIG. 4 shows an embodiment of model fitting;

FIG. 5 shows a flow chart of a further process for processing FMCW signals.

### DETAILED DESCRIPTION OF EXEMPLARY EMBODIMENTS

By way of illustration an embodiment a frequency modulated continuous wave radar FMCW will be described. However, it should be appreciated that any type of radar processing may be used that, like FMCW, can be used to measure properties of radar reflections from a target (e.g. a human body) and resolve the reflections from different distances (i.e. resolve range) and frequencies. For example a pulsed radar may be used that is configured to generate pulses at a plurality of frequencies, and detect reflections at these frequencies. As another example a wideband pulse radar may be used that is configured to transmit wide band pulses, receive reflections and analyze different frequency components of the pulses.

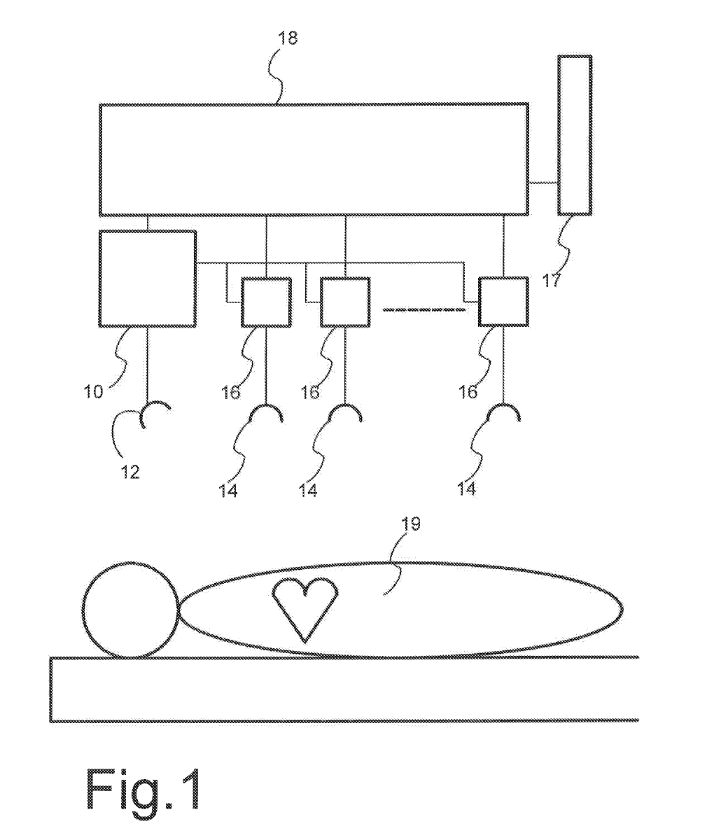

FIG. 1 shows a radar system. The system comprises a signal generator 10 coupled to a transmission antenna 12, one or more reception antennas 14 coupled to an output of signal generator 10, each coupled to an input of a mixer 16, a signal processing system 18 coupled to the output or outputs of mixers 16 and a display 17 coupled to signal processing system 18. Mixers 16 have further inputs coupled to signal generator 10. Although reception antennas 14 are shown as distinct antennas, it should be appreciated that at least part of the reception antennas may double as transmission antennas 12. Although three reception antennas 14 are shown by way of example, it should be understood that another number of antennas may be used, for example a much larger number than three. Although not shown, the system may comprise additional elements such as amplifiers or receivers coupled between reception antennas 14 and mixers 16, isolators etc. Although only a single transmission antenna is shown, it should be noted that more than one may be used. The number of receiver antennas will be represented by N_{R} and the number of transmission antennas will be represented by N_{T}. signal processing system may be a single computer or a system of processing circuit. It may be a programmable processing system, programmed with instructions to make it perform the operations described herein. In the embodiment with a programmable system, actions described herein for the signal processing system mean that the instructions are configured to make the signal processing system perform these actions. The program or programs comprising these instructions may be provided on a computer readable medium, like a magnetic or optical disk or a (non-volatile) semi-conductor memory.

FIG. 1a shows an embodiment wherein signal generator 10, comprises band specific generators 100 for a plurality of frequency bands. Although three band specific generators 100 are shown by way of example, it should be understood that two or more than three may be used instead. In the figure mixers 16 have been replaced by mixer systems 160, which are coupled to the different band specific generators 100 in parallel. Each mixer system 160, mixes received radar reflections with sweep signals from each of band specific generators 100. FIG. 1b shows an embodiment of a mixer system 160, comprising a plurality of mixers 164 for respective bands and a splitter 162 coupled between an input for a reception antenna (not shown) and mixers 160. Although one input for one reception antenna is shown, it should be appreciated that instead different antennas may be used for different frequency bands, coupled to respective ones of mixers 164.

Although FIG. 1a shows an embodiment with a plurality of band specific generators 100, it should be appreciated that instead a single generator may be used that is swept in or through a plurality of frequency bands e.g. over one or more ranges within 2-80 GHz. In this case a plurality of mixers may not be needed. Use of a plurality of generators and mixers has the advantage that they can be used simultaneously, so that measurements can be performed over a larger number of frequencies within a shorter time interval than with a single frequency at a time FMCW radar

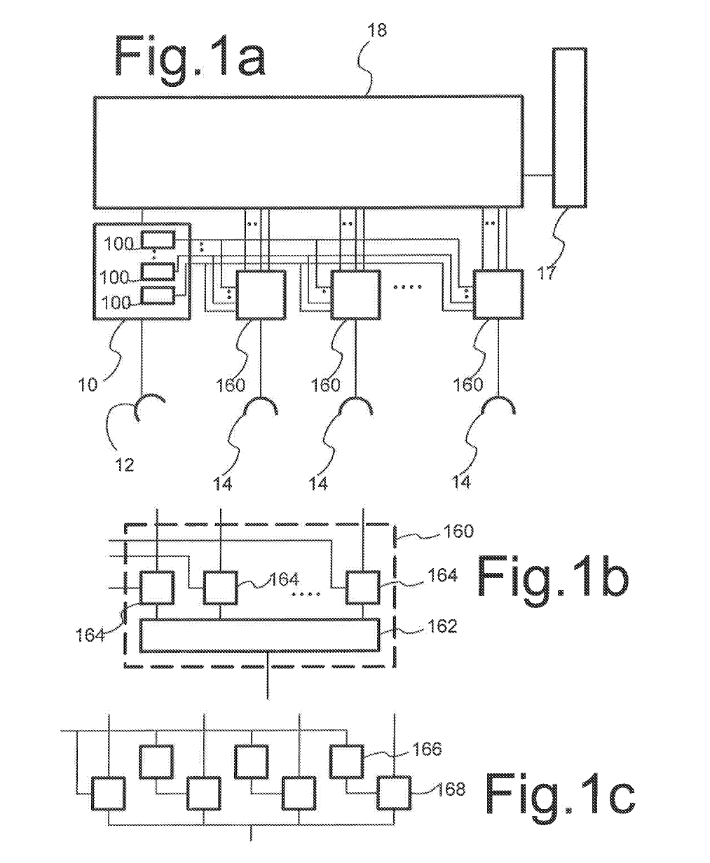

In an embodiment the radar system is a frequency modulated continuous wave (FMCW) radar system. FMCW radar is known per se. In an FMCW radar, signal generator 10 is a frequency modulated signal generator, configured to generate a signal with a frequency that ramps up or down as a function of time repetitively in a series of sweeps, as illustrated in the example of a plot 20 of frequency versus time in FIG. 2. As the sweep has a period duration Tₜ. The radar signal need not be transmitted over the entire period. The part of the period wherein it is transmitted is called the chirp, which has a chirp duration T_{c} that may be smaller than the period duration Tₜ. The signal is said to be continuous because it continues over the transmitted range of frequencies in the sweep, although of course it may be switched off outside sweeps. An FMCW “time-sample” corresponds to the measured responses from a set of such sweeps. In an example wherein the sweep duration is 0.5 msec and 32 sweeps are used in an FMCW time-sample, the time sample corresponds to a 16 msec time interval. Over a heart beat cycle of about 1 second about 60 of such samples are available.

The baseband description of the generated signal by the FMCW hardware is the complex signal:

sₜ(t)=S₀e^{jφ}^{ₜ}^{(t)},

Wherein S₀ is a constant amplitude j is a root of minus one and φₜ(t) is the generated signal phase with period Tₜ, chirp with duration T_{c}. The generated phase is related to the generated angular frequency according to:

$${ω}_{t} ( t ) = \frac{\partial {φ}_{t} ( t )}{\partial t} .$$

In FIG. 2, graph 20 shows the frequency as a function of time. Integration of the frequency gives the transmitted phase. In the case of sweeps with a linearly varying frequency, integration of the linearly varying frequency for the time interval 0≦t≦T_{c} gives the transmitted phase for a perfect linear sweep:

φ_{g}(t)=α₀+α₁t+α₂t^{2.}

Herein α₀, α₁, α₂ depend on the transmitter but are independent of the reflecting object. The carrier frequency ƒ₀ is defined by the constant term ƒ₀=α₁/(2π) and the modulation bandwidth B is related to the slope and duration of the frequency modulation B=α₂T_{c/π.}

In FIG. 2 graph 22 (dashed) shows the frequency of a return echo from an object point, as received as part of the signal at a reception antenna (the return echo from a point is received only in intervals with a length of the chirp duration). Compared to the transmitted signal, this return echo has a frequency shift, and is therefore called a beat signal. In the case of reflection from spatially distributed object points, i.e. from a distributed object, the received signal has a spectrum of frequencies, different frequency ranges corresponding to different ranges of radar signal travel time from the transmitter antenna to an object and back to a receiver antenna. The return echo with delay τ covers the distance range from the transmitter antenna rₜ to the object and the range from the object to the receiver antenna rᵣ.

$$τ = \frac{{r}_{t} + {r}_{r}}{c} .$$

When the object point does not move during the transmission, the received signal on the antenna is:

sᵣ(t)=sₜ(t−τ).

The received signal in the receiver is mixed with the generated signal, which gives the beat signal:

S_{b}(t)=sₜ(t)sᵣ^{*}(t).

If we ignore the phase shift of the target reflection, the phase of the beat signal is the substitution of equations:

φ_{b}(t)=φₜ(t)−φᵣ(t)=b₀−b₁t.

With b₀=α₁τ−α₂τ², and b₁=α₂τ. In the equation for b₀, α₁ is much larger than α₂ so that b₀ approximately reduces to:

b₀=α₁τ.

This equation shows that the phase shift is independent of the target phase shift. Here it should be remembered that α₁ and α₂ depend on the transmitter but not on reflecting object. The reflecting point enters through the delay τ, which corresponds to the travel time from the transmitting antenne to the receiving antenna via reflection from the object point, i.e. the object range. Given b₀ or b₁ the delay can be determined. The beat frequency ƒ_{b} is related to the object range:

$${f}_{b} = \frac{1}{2 π} \frac{\partial {φ}_{b} ( t )}{\partial t} = \frac{{b}_{1}}{2 π} = \frac{{a}_{2} τ}{π} .$$

Substitution of the amplitude modulation due to reflection gives the received signal component due to a reflecting point:

s_{b}(t)=Aₖ(t)e^{j[b}^{₀}^{+b}^{₁}^{t]}.

Herein Aₖ(t) is a reflection coefficient of an object point and k indexes different object points. The total received signal is a sum (integral) of signals from different points k.

The radar transmits sweep with Sweep Repetition Interval Tₜ. Succeeding sweeps are sampled with Tₘ=mTₜ where Tₘ is the start time of the m-th sweep. The time delay τ changes from sweep to sweep due to the target motion indicated with τ(t). The complex beat signal for succeeding sweeps is:

s_{b}(mTₜ+t)=Aₖ(mTₜ+t)e^{j[b}^{₀}^{+b}^{₁}^{mT}^{ₜ}^{+t)],}

with

b₀=α₁τ(mTₜ)−α₂τ(mTₜ)²,

b₁=α₂τ(mTₜ).

These coefficients depend on the sweep number m. From this dependence, the Doppler speed variation and range of a reflecting point can be inferred. The b₁ term can be used to give the location range of the object point. The b₀ term of the equation gives an Doppler speed variation of the signal.

An FMCW time-sample comprises a set of radar measurements that can be represented as a function of three dimensions. A first dimension is the “fast time” t, which is represents time points relative to the start of the sweep from which the signal was obtained. A second dimension is a “slow time”, which corresponds to a sweep count nₛ: the sequence number of the sweep in the series of sweeps. A third dimension represents the reception antenna 14 that received the reflected signal or all transmitter/receiver antenna combination that resulted in the signal. Because of the three dimensions the measurements are referred to as a (measurement) cube.

Spectral analysis (Fourier transform) of the dependence of the radar measurement on fast time may be used to resolve reflection contributions from different location ranges, and spectral analysis of these contribution ranges may be used to resolve contributions from different Doppler shift (speed) ranges. Combinations of signals from reception antennas at different antennas may be used to resolve contributions from different angles towards the object. In the case of a 1D antenna configuration azimuth angles can be resolved. In the case of a 2D antenna configuration the result azimuth-elevation angles can be resolved.

Thus, after conventional FMCW processing the measurements of the cube for an FMCW time-sample comprises a set of can be transformed to results as a function of range, speed and direction, i.e. as a range-speed-direction data cube (although the direction may in fact be a two-dimensional variable, the arrangement of measurements will be referred to as a “cube” in this case as well). The resolution of the range dimension depends on the bandwidth B of the sweep, which is necessarily more limited in lower frequency ranges. The resolution of the speed-dimension of this cube depends on the number of sweeps used. The resolution of the direction dimension of this cube depends on the number of reception antennas used.

### Six Port Radar

It is known to use so-called six port radar sensing for remote respiration rate and heartbeat vital-sign monitoring. See G. Vinci et al., “Six-Port Radar Sensor for Remote Respiration Rate and Heartbeat Vital-Sign Monitoring,” IEEE Transactions on Microwave Theory and Techniques, 2013, ImprovementVitalSign.

In six port radar, collected signals are power B₃, B₄, B₅, and B₆ obtained by mixing in with four different phase offsets. The resulting baseband signals can be handled like differential I/Q signals as:

Z=I+jQ=(B₅−B₆)+j(B₃−B₄)

and

Δσ=φ₁−φ₂=arg{Z}.

Distance information can be extracted from the baseband voltages by equation

$$d = \frac{1}{2} \frac{Δσ}{2} {λ}_{RF} .$$

Herein λ_{RF} is the wavelength of the transmitted radar signal at the frequency that is used for this computation. A wideband frequency source may be used to synthesize a narrow pulse in order to accomplish reflection coefficient measurements over a large frequency band. In the art six-port measurements with baseband power detectors are used. We use down converters to baseband followed with analog to digital conversion if six port radar sensing used. The resulting digitized signals give four measurements cubes.

When a six-port radar is used, each mixer may comprise a plurality of mixing circuits for respective phases, and a plurality of outputs to the signal processing system. FIG. 1c shows an example of a mixer with a plurality of mixing circuits 168 and phase shifters 166 configured to shift the frequency generator signal by respective amounts of phase shift for six port reception. Alternatively, or in addition, a multi-phase frequency generator may be used to provide signals with respective amounts of relative phase shift.

### Multiband FMCW Radar

Frequency modulated signal generator 10 is configured to generate FMCW signals in a plurality of frequency bands, the frequency separation between the bands being much larger than the bandwidths of the bands Bₖ where index k indicates the radar bands. Exemplary bands are in the 2.4 GHz, 24 GHz, 60 GHz and 76 GHz ranges, and the bandwidth (sweep range) in each of these ranges may be up to 10% of the frequency used (e.g. 200 MHz sweep band for a 2.4 GHz signal). Besides these separated bands it is also possible that the radar has one continuous band which can be divided into a number of separate bands e.g. from 2.4 GHz to 18 GHz.

For generating FMCW signals in a plurality of frequency bands frequency modulated signal generator 10 may comprise a plurality of oscillators for the respective bands. The signals for different bands may be transmitted successively or concurrently. The may be transmitted simultaneously, in which case a plurality of mixers 16 may be used for each reception antenna, each to mix the received signal with the FMCW signal for a respective frequency band (optionally after amplification in that band).

In another embodiment concurrent transmission may be realized by interlacing sweeps in different frequency band. Also in this case a plurality of mixers 16 may be used for each reception antenna, but this is not indispensable.

Band width Interpolation (BWI) may be applied to combine the FMCW signals from different frequency bands during the interval of an FMCW time-sample. This can be used to remove the lower limits of the range resolution associated with the bandwidths of the individual sweeps.

In the fast time domain, the BWI combined signal corresponds to the fast time dependence of a response signal that would virtually be obtained by transmitting an FMCW radar signal of which the radar frequency is swept over a frequency range that includes all bands, receiving its radar reflection and mixing that reflection with the FMCW radar signal. Thus, this virtually obtained response signal has a longer time duration than the actually measured response signals. Each time point in the response signal can be associated with a frequency and phase, which is the frequency and phase of the sweep of the virtually transmitted radar signal at that time point (distinguished from the frequency and phase of the BWI combined reflection signal at that time point). For time points that correspond to frequencies in the bands that were used in the measurements, the BWI combined response signal corresponds to the measured response signals obtained by reception of radar reflection and mixing when those frequencies were transmitted with the corresponding phase. For other time points the BWI combined signals are interpolations. BWI combines all band coherently in range, speed and direction.

In embodiments of BWI, the fast time dependence of the BWI combined signal need not actually be computed: instead the Fourier transform of the BWI combined signal may be computed directly form the Fourier transforms of the FMCW responses in the different frequency bands.

Because of the longer time duration, the Fourier transform of the fast time of the BWI combined response signal has a higher frequency resolution. Effectively the effect of a much longer sweep from the lowest frequency of the lowest band to the highest frequency of the highest frequency band The resulting range-speed-direction data cube has improved target resolution, improved speed resolution and improved direction resolution.

Application of Radar Signals to the Human Body

When a radar signal is transmitted to a human body, the received signal from the radar is a sum of reflections from objects including at least a part of the human body and its contents. Possibly surrounding objects also contribute to reflections. Different parts of the chest are at slightly different distances from the radar antennas rₖ where k is an index of the antennas. The reflection coefficients of a point on the object changes in time due to the blood perfusion this is indicated in the Aₖ(t). The total signal is the combination of all individual contributions.

The reflection coefficients Aₖ(t) depend on the frequency range of the radar. At high frequencies such as 60 GHz and 76 GHz, almost all radiation is reflected from the skin, whereas at lower frequencies such as 2.4 GHz there is also a considerable contribution of reflections from structures within the human body. Within the bands of the individual sweeps modulation by the amplitude of Aₖ(t) has been found to be negligible.

The highest frequency bands such as 60 GHz and 76 GHz provide the most accurate resolution of range (distance). Because almost all radiation is reflected from the skin this can be used only to determine the location of the skin on the chest (fast time), movement of the chest (slow time) and changes of blood perfusion of the skin on the chest (reflection coefficient). The radar signal in the lowest frequency bands, such as the 2-10 GHz e.g. around 2.4 GHz, also depends on the reflection coefficients of internal tissue, because all radiation is reflected from the location of the skin at these frequencies. However, accurate resolution of range is less accurate at these frequencies.

The following phenomena can observed using radar:
Chest movement
Internal heart movements
Variation of the skin reflection coefficient.

The first step is determination of the chest motion, followed by use of this motion as a reference for determining of variation of the reflection coefficient of the skin. This can be done with the aid of a radar system that uses multiple bands (2-10 Ghz band, 24Ghz band, 60 GHz band or 76 Ghz band). The high frequency bands (24 GHz band, 60 GHz band or 76 GHz band) have no penetration and the and only skin/surface movement is measured. The optimal frequencies can be determined experimentally. Very accurate estimation of surface movement is possible with these high frequency bands. This motion estimator is used to distinguish between internal movement caused by the heart and variation of the reflection coefficient caused by the chest. What remains after removal of surface movement are only the contributions caused by internal heart movement and variation in the reflection coefficient (which can vary between different bands).

The measurements using high frequency radar of e.g. 60 Ghz are translated into measurements of low frequency band radar. The difference is the required signal, that shows only the internal reflections and modulations of the reflection coefficient. If the high frequency bands also show a variation of the reflection coefficient this will give a variation which depends on the Δ┌ in that band. The variation Δ┌ of the low frequency radar band is larger than that in the high frequency bands. The difference is measured. When internal reflections have been determined with the heart model, the remainder is the variation of the reflection coefficient.

Residue response=Measurement in low frequency band−measurement in high frequency band (internal motion and change of reflection coefficient)

Comparison of the Signals from Multiple Bands

Heart measurements with radar show differences between amplitude modulation and phase modulation. The amplitude modulation is caused by the variation of the skin reflection. The phase shift can be a property of the human body. We have observed that the phase shift is approximately equal over the different bands. Differences are due to different position of the radar antennas. The amplitude variation are combined over different bands.

Possibility to Separate Shift and Amplitude Modulation from Each Other

The amplitude modulation is almost independent of the aspect angle.

The multiband radar systems observes this amplitude modulation in all radar bands. Suppose we measure straight in front of a person and the reflection coefficient has a sinusoidal variation as a function of angle. When the receivers are close to each other there is almost no amplitude variation between the measurements. There is phase variation for example if the antennas are half a wavelength apart this is maximally half a wavelength. In time the amplitude variation occurs at same time points.

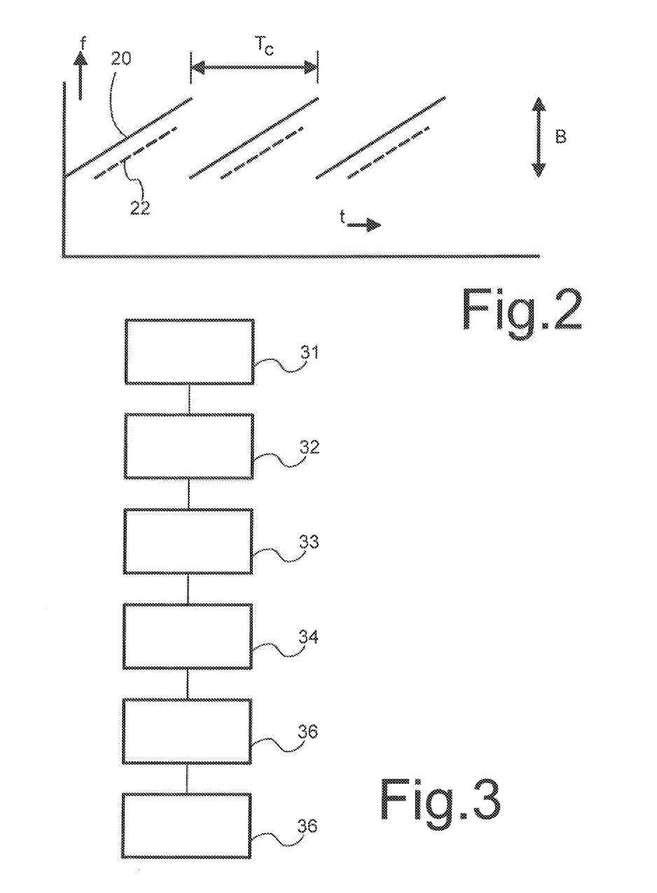

FIG. 3 shows a flow-chart of multi frequency signal processing. In a first step 31 signal processing system 18 captures reflected radar signals for an FMCW time-sample from a plurality of frequency bands including a relatively higher frequency band or bands wherein only skin/surface movement is measured and a relatively lower frequency band or bands wherein not only skin/surface movement is measured. First step 31 is repeated for a series of an FMCW time-samples during a time interval that is at least as long as needed to cover one heart beat cycle. In a second step 32 signal processing system 18 estimates chest motion. Very accurate estimation of surface movement is possible with the higher frequency bands. In a third step 33 signal processing system 18 determines variation of the reflection coefficient of the skin using the estimated chest motion as a reference for this.

In a fourth step 34 signal processing system 18 uses the estimated chest motion to distinguish between internal movement caused by the heart and variation of the reflection coefficient caused by the chest. Signal processing system 18 translates the measurements obtained using a higher frequency radar of e.g. 60 Ghz into measurements of low frequency band radar. This defines a residue response, which is a difference between the measurement in a lower frequency band and the translated measurement from the higher frequency band. What remains after removal of surface movement are only the contributions caused by internal heart movement and variation in the reflection coefficient, which can vary between different bands. Thus, the residue defined by the difference represents internal motion and change of reflection coefficient.

The difference shows only the internal reflections and modulations of the reflection coefficient. If the high frequency bands also show a variation of the reflection coefficient this will give a variation which depends on the Δ┌ in that band. The variation Δ┌ of the low frequency radar band is larger than that in the high frequency bands. The difference is measured. When internal reflections have been determined with a heart model, the remainder is the variation of the reflection coefficient.

In a fifth step 35, signal processing system 18 performs inverse modeling to estimate a value of one or more parameters of the heart. As is known per se, inverse modeling makes use of a forward model, which expresses predicted radar signals for a series of FMCW time-samples as a function of one or more parameters of the heart, to determine values of the one or more parameters that result in a prediction that corresponds to measured radar signals. This is also called model fitting, which may comprise performing a search for values of the one or more parameters that minimize a measure of the difference between the predicted and measured radar signals. The parameters may include hart beat frequency and phase and optionally heart scale or other parameters.

In a sixth step 36, signal processing system 18 generates an image of the heart according to these one or more parameter values and causes display 17 to display this image. Alternatively, or in addition, signal processing system 18 may cause display 17 to display one or more characteristic values of the heart computed based on the estimated values of the one or more parameters and/or additional measured parameters such as heart rate.

As noted, similar measurement cubes may be obtained using other types of radar than FMCW radar, like multi-frequency pulsed radar and wideband pulsed radar.

### Model Fit

(1) Search for the best fit with the geometric model. The geometric model is available. It is possible to use steering features in the fit. Among others, steering features are features which describe a change of temporal development, other trajectories e.g. dimensions of model components (leg length, step size), principal components of the heart for example. This is a low dimensional geometric model. For example in the case the heart the heart volume and in the case of human motion the step frequency and the step length.

(2) Calculate (a) the Taylor expansion at the minimum wherein the grid points are the variables, (b) by the approximation the fit error is related to errors in the grid points. The problem now becomes a linear problem and is a remapping of the original model parameters to grid points. The possibilities of remapping are dependent on the model. In the case of a heart model a transition from a frequency/volume model to the grid model can be made.

(3) By means of a least square solution the model error is translated to displacements of grid points. The solution of this problem is a minimization under constraints.

(4) The grid displacements are mapped with the geometrical model. The geometric grid is displayed to the observer (imaging to user).

(5) Optionally the residue projections can be averaged over time (extra optimization step).

(6) The entire first model can now be left alone and only the grid model is adapted. This minimization will cost a lot of time and the constraints must be selected properly. The fit can be performed also because good initial conditions are available (This is the fit of the grid model)

(7) The entire optimal model is presented to the user.

The minimization in step 1 may require a high computation time. The solution is a feature fit instead of a model fit. This works as follows. The model is available and by means of the model radar measurements are generated. A feature extraction process is applied to the generated measurements, giving the features associated with these parameters Fi. This is done for all model parameter options. After this step we have a collection of features for all model settings Fi for 1<i<M. Subsequently the measurement is performed and the features F of this measurement are determined. Subsequently it is determined which Fi is closest to F. At the end the error must still be determined, but it may be possible to avoid this. We then already have a very good estimate of the fit parameters. The Jacobean of the Taylor expansion can be computed in advance. In the model of the person the length of the limbs and an angle rotations can be varied to obtain an optimal fit to the model. This is not the case for the heart model.

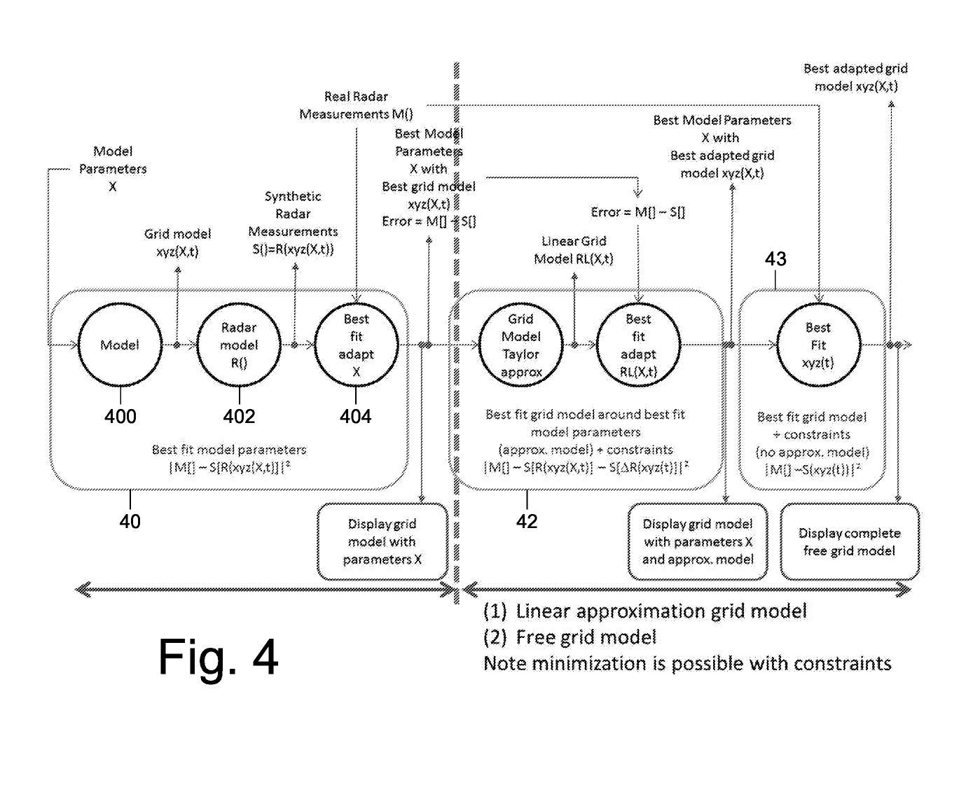

FIG. 4 shows the steps involved in an exemplary embodiment of model fitting. In a first fitting process 40 signal processing system 18 fits parameter values. The first fitting process uses the radar measurements M (preferably obtained by removing surface movement) as input. A model parameter X, which may be a vector of parameters, is used as input to a model 400, which defines a grid model of the heart, which expresses the three dimensional locations xyz(X,t) of a grid of elements of the heart as a function of time t and the value of the parameter X. The elements preferably have radar response properties specified by the model. In an embodiment, the elements are volume elements of the heart with specified radar response properties at three dimensional locations and the model specifies the position of the elements as a function of temporal position in a heart beat cycle. Using heart beat cycle frequency and phase as parameters this defines the position of the elements as a function of time. Additional parameters may be a scale factor to be applied to the positions and/or parameters that express unequal displacements in different principal components of the heart of the heart corresponding to possible deformations of the heart. The grid model produced using the model 400 is used as input to a radar model 402.

Radar model 402 expresses a synthesized radar signal S as a function of the grid model. For example, if the model defines variation of radar transmission properties as a function of position, reflection can be computed using standard electromagnetic theory. In first fitting process 40 signal processing system 18 executes a parameter adaptation process 404 wherein signal processing system 18 performs a search for value for X that minimizes an error measure dependent on the difference between the measured values M and a synthesized radar signal S resulting from that parameter value X. The value that results from this search is called the best fit parameter.

In a second fitting process 42, signal processing system 18 computes a best fit grid model around the best fit grid model obtained by fitting the parameter X. Second fitting process 42 makes use of a Taylor approximation of the grid model 420, i.e. a model wherein the effect of displacements ΔR(xyz(t)) of the elements of the heart on the resulting radar signal are modeled as linear changes in the synthesized radar signal S. In second fitting process 42 signal processing system 18 executes a displacement adaptation step 422 wherein signal processing system 18 performs a search for a combination of values for ΔR(xyz(t)) that minimizes an error measure dependent on the difference between the measured values M and a synthesized radar signal S resulting from adding these displacements ΔR(xyz(t)) to the grid model. This search may be performed using matrix inversion and/or solution of linear equations, which makes it a relatively fast process. In an embodiment, constraints may be imposed on displacements ΔR(xyz(t)) that are allowed in this search. For example a constraints imposing spatial continuity may be imposed by adding a sum of squared gradients to the error measure, the sum (integral) being taken as a function of position in the heart. Effectively, this models elasticity.

In a third fitting process 43, signal processing system 18 computes a best fit grid model, i.e. a set of locations xyz of the elements as a function of time without Taylor approximation. In third fitting process 43 signal processing system 18 performs a search for a combination of values for the grid points xyz(t)) that minimizes an error measure dependent on the difference between the measured values M and a synthesized radar signal S resulting from these grid points xyz(t), using the grid model according to the parameters found in first fitting process 41 and the displacements found in second fitting process 42 as starting point. As may be noted, the grid points that result of this search are not constrained by the model M, although it may be that the starting point derived from the model may have the effect of selecting from a plurality of local minima of the error measure that can be found. The selection of the starting point reduces the time needed for the third fitting process.

As shown in the sixth step of FIG. 3, an image of the heart according to the gird model may be generated and displayed. This may be applied to the grid model resulting from first, second and/or third fitting process 41, 42, 43.

### Embodiments of Determination of Heart Parameters.

Embodiment 1: Multi-band radar systems to separate internal and external reflections from each other. Measurements in three bands may be used for example and by combining these the internal reflections may be determined in a low frequency band (around 2.4 GHz) This is followed by the model fit as described in EP 2368492A2 but with the additions of a determination of variation of the reflection coefficient.

Embodiment 2: Multi-band radar systems to separate internal and external reflections from each other and to combine this with an appropriate estimate or measured of the variations of the reflection coefficient. The objective is to estimate the reflection coefficient of e.g. the thigh bone as well as possible by compensating for the reflection of the skin. This can be combined with embodiment 1.

Embodiment 3: Use of a multiband-six port radar that is able to determine the reflection coefficient at a distance. This can be combined with embodiment 1 and 2.

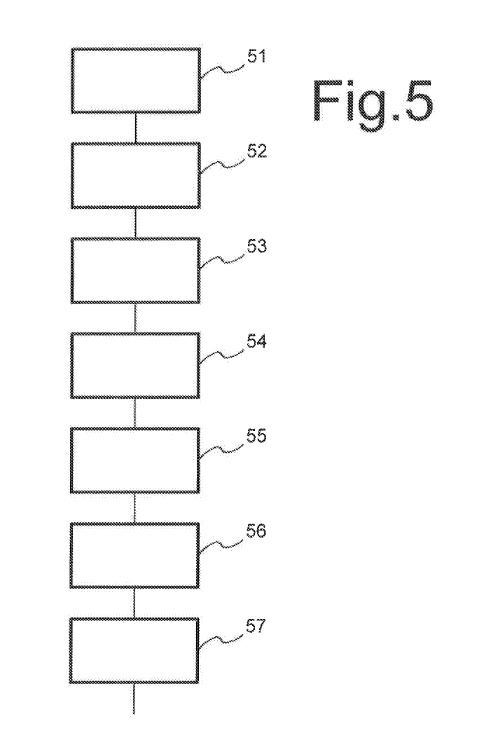

FIG. 5 shows a flow chart of a further process of operation of signal processing system 18 for processing FMCW signals. In a first step 51, signal processing system 18 obtains FMCW radar responses for a plurality of M bands. In a second step 52 signal processing system 18 calibrates the FMCW radar responses with a range-calibration-curve. A range-calibration-curve may be used that relates the measured signal strength to a normalized strength. A range-calibration-curve may be used that is determined by measuring the signal strength measured from a standardized object, e.g. a corner reflector, to predicted responses for that standardized object. This results in fast time-slow time antenna position cubes of measurements for a plurality of frequency bands.

Alternatively, similar steps may be performed using other types of radar than FMCW radar, like multi-frequency pulsed radar and wideband pulsed radar, to produce similar cubes.

In a third step 53, signal processing system 18 translates these cubes to range-speed-azimuth responses. Algorithms for doing so using Fourier transforms are known per se from FMCW radar processing. In a fourth step 54, signal processing system 18 applies Band Width Interpolation (BWI) to the range-speed-azimuth responses. Optionally, a form of BWI may be applied directly to the fast time responses. In this case third step 53 may be executed later. BWI combines all bands coherently in range, speed and direction. The range-speed-direction data cube that results from applying BWI has improved target resolution, improved speed resolution and improved direction resolution.

The process up to and including fourth step 54 comprises (1) calculating individual range-speed-direction data responses (2) calculating combined range-speed-direction data response from a plurality of frequency bands. Thus for each an FMCW time-sample combined range-speed-direction data response is produced. These steps may be repeated for a series of an FMCW time-samples. Similarly, they may performed for multi-frequency time samples from other types of radar, like multi-frequency pulsed radar and wideband pulsed radar.

In an optional fifth step 55 signal processing system 18 tracks peaks in the responses in the range, speed and direction cube obtained by BWI through a series of FMCW time-samples, or other type of multi-frequency samples. In the case of heart observation, peaks with different properties can be associated with different physical features.

Fifth step 55 comprises (3) detecting peaks in the responses as a function of range-speed-direction (4), and selects tracks of the detected peaks through successive FMCW or other time-samples for the range, speed and direction cube obtained by BWI. In the tracks only peaks are retained that lie on at least partly continuous tracks or extrapolations of continuous track parts. Isolated peaks. Optionally, signal processing system 18 may also detect peaks in the range, speed and direction cubes for individual bands, track peaks from the different bands and retain tracks from individual range-speed-direction responses only if they match with tracks in the combined response.

As a function of range (distance to the radar), the closest detected response peak, which will be called the first detection, should be the response from the skin of the chest. Tracking can be used to eliminate “false” first detections that do not correspond to the chests and/or to identify weak responses of the chest at locations predicted by the track. The first detection may be used to determine the location of the surface of the chest. The heart results in the closest moving response, i.e. the closest response with a Doppler shift above a predetermined threshold. All other objects are stationary objects and give zero responses for non-zero Doppler speeds.

In a sixth step 56 signal processing system 18 uses the location of the chest surface, as determined from the first reflection in different directions, to remove the peaks associated with the response from the chest surface from the original signal. When fifth step 55 is used, peaks that correspond to the selected track at closest range may be used as first detections.

In an embodiment sixth step 56 may implement peak removal using measured properties of a detected reflection peak of the first reflection to select parameters of a predetermined function that represents a modeled signal that corresponds to the reflection peak. The measured properties of a detected reflection peak may include peak amplitude. Amplitude change rate (ASR) and frequency change rate (FCR) may be used for example. The modeled signal may be represented in the fast and slow time domain and/or per antenna for the respective frequency bands for which measurements have been obtained. Alternatively a full or partial frequency domain representation (distance range, Doppler shift and/or direction) may be used. The modeled signal is subtracted from measured signals in at least one of the frequency bands and/or in the combined signal produced by BWI. In an embodiment, this is done in the time domain, that is by subtraction the modeled signals for the respective sweeps for a frequency band from the measured response during each sweep in that frequency band.

Sinusoidal modeling modelling may be used, as described by Abe et al. in two reports “Design Criteria for the Quadratically Interpolated FFT Method (I): Biasdueto Interpolation,” and “Design Criteria for the Quadratically Interpolated FFT Method (II): Biasdueto Interfering Components,” in tech reports dates Oct. 13, 2004 from Center for computer research in music and acoustics department of music, Stanford University. Abe describes the Quadratically Interpolated FFT (QIFFT) method for estimating sinusoidal parameters from peaks in spectral magnitude data. The sinusoid signal is approximated with a first order AM and FM. In this case the modeled signal is

Exp(λ+αt+j(φ+ωt+βt²))

In the case of an FMCW signal, the values of time “t” can be associated with radar frequencies and phases (not otherwise in the formula) that are reached at these time points in the sweep. The value of the modeled signal corresponds to the response obtained after mixing with the radar signal at that time point. In the case of a bandwidth interpolated FMCW signal, the values of time “t” correspond to a wide range of radar frequencies.

The modeled signal has parameters called λ: the log amplitude, α: amplitude change rate, φ: phase co: frequency and β: frequency change rate. When the Fourier transform in the fast time domain of the bandwidth interpolated signal corresponds the Fourier transform that has been weighed as a function of time with a Gaussian function with variance ½p (G=exp(−pt²)/norm), such a time dependent signal results in a peak in the Fourier transform at a frequency of ω+αβ/p with an amplitude of which the logarithm is λ+α²/4p−log(1+(β/p)²) and a phase at the peak of φ+α²β/4 p²+0.5atan((β/p). The first derivatives of the phase of the peak is −α/2p and the second derivatives of the amplitude and phase are −p/2(p²+β²) and −β/2(p²+β²).

By means of these relations the parameters λ, α, φ, ω, β and optionally p can be estimated from the amplitude and phase of the peak and their derivatives in the measures signal cube. Using the estimated parameters the modeled signal can be computed for any time point and subtracted from the measured reflection signal. They can be subtracted from the measured and mixed reflections for individual bands by substituting the time values that correspond to the frequencies and phase values in the frequency sweep for that band.

It should be noted that the modeled signal with parameters λ, α: φ, ω: and β is only one embodiment. Other forms of modeled signal may be used, of which parameters may be estimated from a peak. Obviously, these may lead to slightly different removals of chest signals, but as long as a significant part of that signal is removed the model results become more reliable. In an embodiment, the subtraction is applied to the processed range-speed-direction data. For this the model signal is converted according to the conversion to range speed direction and then subtracted. This minimizes artefacts. Alternatively, the subtraction may be to the radar response before full conversion to range-speed-direction, using a correspondingly different version of the modeled signal. In this case the conversion of the signal in a band to range-speed-direction of third step 53 may be performed, or at least completed after subtraction.

The chest body and blood perfusion modulation and the chest motion are available in more than one of the different frequency bands. All these bands have these responses. The high frequency bands has no penetration deeper into the human body. The amplitude and phase modulation may differ between different frequencies. The amplitude modulation is caused by the variation of the skin reflection. The phase shift can be a property of the human body, dependent on blood perfusion. We have observed that the phase shift is approximately equal over the different bands. The estimated amplitude and phase modulation give the chest movement and the blood perfusion. Signal processing system 18 may be configured to cause display 17 to display the individual signals.

In an optional seventh step 57 signal processing system 18 determines a temporal variation of a reflection coefficient associated with the skin, from signals received in a succession of FMCW sample intervals. The reflection coefficient varies with time during a heart cycle due to increases and decreases in blood perfusion. Therefore the reflection coefficient can be used as a measure of blood perfusion. In particular, the variation of the phase of the reflection coefficient can be used as a measure of blood perfusion. Step 57 comprises selection of a frequency band from which the reflection coefficient will be determined. Signal processing system 18 selects this band from the higher frequency bands wherein little or no skin penetration occurs, subject to the condition that a track of first detections is detected in that band. From at least one response peak along that track in the selected band, signal processing system 18 determines the amplitude and phase for successive FMCW time intervals during a heart cycle. After phase and amplitude calibration the amplitude and phase represent the reflection coefficients. The range of variation of the phase and amplitude calibration during a heart cycle may be used to output an indication of blood perfusion. A six port radar measurement (e.g. obtained with a mixer as shown in FIG. 1c may be used to obtain more accurate measurements of the reflection coefficient.

Band selection may comprise comparing the signals from different bands with each other after the removal of chest motion effects. Comparison gives the differences in the bands due to other illuminated parts in the radar beam or other heart characteristics for example the reflection coefficients. The comparison gives chest reflection and perfusion modulation for different bands.

In an embodiment signal processing system 18 may perform adaptions if the amplitude and phase or if the time in different bands is not aligned. Assume the higher bands gives the synthetic signal presented with s_{r,h}(t) and the lower band the range signal s_{r,l}(t). The high band signal is a digital representation and known for each time. The lower band signal is sampled and not known. A non linear least squares fit may be used to compute the matching parameters A, t_{d} that minimize

$$( {\sum }_{all} {( {s}_{r , l} ( t ) - {As}_{r , h} ( t - {t}_{d} ) )}^{2} )$$

Herein the sum is taken over all time points. In an embodiment background may be subtracted. Background is a stationary signal that is constant during the measurement time. Processing system 18 may remove background with a complex linear least fit line. A window length of approximately one breathing period may be used. This compensation may be applied to each radar band.

The results of the process described by means of FIG. 5 may be used as measured signal M for the heart model fit.

Although an example has been elaborated for measuring heart parameters or heart imaging, it should be appreciated that similar techniques may be applied to other parts of the body. For parts of the body that do not deform cyclically, like limbs that contain bone structures a less complex model than that of the heart may suffice.

1. A method of measuring internal body motion using radar, the method comprising
measuring radar reflections using a range of frequencies that includes a higher frequency band and a lower frequency band;
estimating chest surface motion using the measurements using the higher frequency band;
removing effects of the estimated chest surface movement from the measurements from the lower frequency band.

2. A method according to claim 1, wherein the higher radar frequency band is a 24 Ghz band, 60 GHz band or 76 Ghz band and the lower radar frequency band is in a 2-10 Ghz band.

3. A method according to claim 1, wherein measuring the radar reflections comprises performing measurements in multiple discrete radar frequency bands.

4. A method according to claim 1, wherein the multiple radar frequency bands include a 2-10 Ghz band, a 24 Ghz band, a 60 GHz band and a 76 Ghz band.

5. A method according to claim 1, any one of the preceding claims, comprising fitting a model of a heart to the measurements.

6. A method according to claim 4, wherein said fitting comprises
fitting parameters X of a geometric model to the measurements;
determining a least square solution of fit errors between the measurements and a Taylor expansion from the grid model obtained with the fitted parameters X as a function of adaptions of the grid model;
determining a further adaptation of the grid model that best fits the measurements.

7. A method according to claim 5, comprising displaying the model obtained by said fitting.

8. A method according to claim 1,
selecting earliest received reflection peaks from the radar reflections of radar transmissions using the higher frequency band;
estimating parameters of the selected reflections peaks;
subtracting a modeled signal that corresponds to a peak with the estimated parameters from the measurements from the lower frequency band.

9. A method to claim 7, comprising tracking the received peaks in response to radar transmissions at successive time points and selecting the earliest peaks from peaks that lie along a closest detected track in a distance range of the radar.

10. A method to claim 7, comprising fitting a model of a heart to the result of said subtraction.

11. A method according to claim 10, wherein said fitting comprises
using inverse modeling from the from the measurements from the lower one the frequency bands to which said subtracting has been applied to determine parameters of a model that defines locations of elements of a human heart as a function of the parameters.

12. A method according to claim 11, wherein said fitting comprises
determining a least square solution of fit errors between the measurements from the lower one the frequency bands to which said subtracting has been applied and a linear expansion of the effect of displacements of locations of elements from the locations defined by said model using the parameters obtained by said inverse modeling;
performing a search for displacements of locations of elements that minimize an error measure for a difference between the measurements from the lower one the frequency bands to which said subtracting has been applied and predicted measurements using said displacements, using the displacements obtained from the least square solution as a starting point for the search.

13. A method to claim 9, wherein fitting of the displacement is performed under constraints that limit or reduce differences between displacements of adjacent elements.

14. A computer program product comprising instructions for a programmable processor that, when executed by said processor will cause the processor to execute the processing of the measured radar reflections claimed in claim 1.

15. A radar system for monitoring internal motion within the chest of a body, the radar system comprising a radar signal generator coupled to a transmission antenna and cause transmission of radar signals in a range of frequencies that includes a higher frequency band and a lower frequency band;
at least one receiver for receiving radar reflections in the higher frequency band and the lower frequency band;
a signal processing system configured to estimate chest surface motion using the measurements using the higher frequency band and to remove effects of the estimated chest surface movement from the measurements from the lower frequency bands.

16. A radar system according to claim 15, wherein the signal processing system is configured to fit a model of a heart to the measurements.
